(19) United States
(12) Patent Application Publication
(10) Pub. No.: US 2010/0121108 A1
(43) Pub. Date: May 13, 2010

(54) PROCESS FOR SYNTHESIZING DESVENLAFAXINE FREE BASE AND SALTS OR SOLVATES THEREOF

(75) Inventors: Jordi Bosch I Llado, Barcelona (ES); Iolanda Chamorro Gutierrez, Santa Coloma De Farners (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona (ES)

(21) Appl. No.: 12/376,247

(22) PCT Filed: Aug. 3, 2007
(86) PCT No.: PCT/IB07/03574

§ 371 Date: Jan. 21, 2010

Related U.S. Application Data
(60) Provisional application No. 60/835,448, filed on Aug. 4, 2006.
(60) Provisional application No. 60/907,541, filed on Apr. 6, 2007.

(51) Int. Cl. C07C 213/00

(52) U.S. Cl. 564/320

(57) ABSTRACT

The invention relates generally to an improved process for manufacturing desvenlafaxine free base and salts or solvates thereof.

### CROSS REFERENCE TO RELATED APPLICATIONS

This application claims priority to U.S. Provisional Application Nos. 60/835,448 (filed Aug. 4, 2006) and 60/907,541 (filed Apr. 6, 2007), both of which applications are expressly incorporated herein by reference in their entirety.

### BACKGROUND OF THE INVENTION

1. Field of the Invention

The invention relates generally to an improved process for manufacturing desvenlafaxine free base and salts or solvates thereof.

2. Relevant Background

Desvenlafaxine (Compound I, below) is an active pharmaceutical substance with an empirical formula of C₁₆H₂₅NO₂ and a molecular weight of 263.38. Desvenlafaxine, which can also be referred to as desmethylvenlafaxine and/or O-desmethylvenlafaxine, is the major active metabolite of venlafaxine, an active pharmaceutical ingredient indicated for the treatment of major depressive disorder.

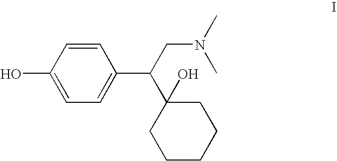

U.S. Pat. No. 4,535,186 discloses the first process for preparing desvenlafaxine. In U.S. Pat. No. 4,535,186, desvenlafaxine is synthesized by the process illustrated in Scheme 1:

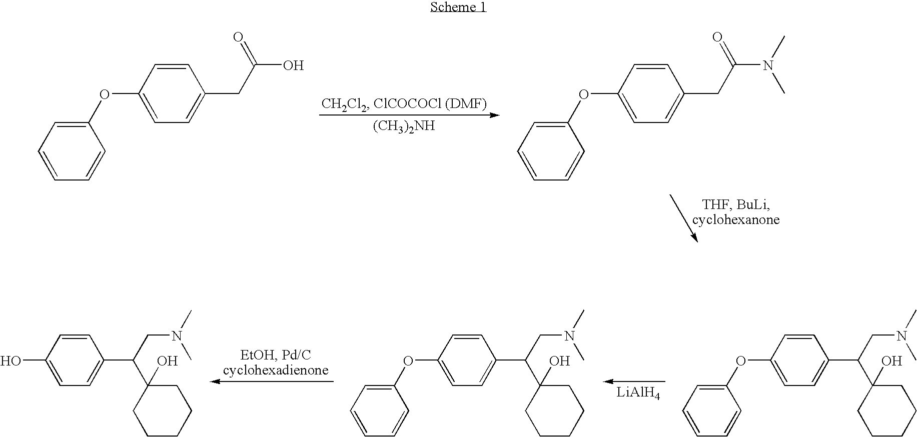

U.S. Pat. No. 5,043,466 describes an improved synthetic process for preparing desvenlafaxine. In U.S. Pat. No. 5,043,466, desvenlafaxine is obtained by using hydrocarbon solvents as the reaction medium during the condensation reaction with cyclohexanone. Additional alternative processes for preparing desvenlafaxine are described in the literature. These alternative processes generally proceed via the dimethylation of venlafaxine.

### DESCRIPTION OF THE INVENTION

The invention relates generally to an improved process for manufacturing desvenlafaxine free base and salts or solvates thereof. In particular, the invention relates to a synthetic process for preparing desvenlafaxine as illustrated in Scheme 2 below. This alternative process includes to preparing desvenlafaxine directly from its N,N-demethylated derivative (Compound III). In this synthetic process, dimethylation is advantageously performed without requiring protection of the phenoxy group but instead by adjusting the reaction conditions.

The N,N-demethylated intermediate can be prepared by O-demethylation of the corresponding intermediate of Compound II. The reaction can be performed using conventional demethylation conditions, such as the use of sodium hydride with diphenylphosphine, the use of dodecanethiol, or the use of L-selectride. Compound III can then be N,N-dimethylated by conventional means, for example by reductive methylation by treatment with formaldehyde and formic acid, or by treatment with formaldehyde and sodium triacetoxyborohydride.

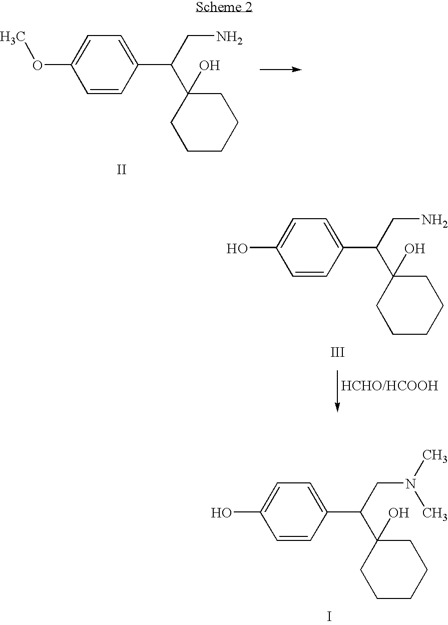

Compounds represented in Scheme 2, employed as raw materials or as intermediates to produce desvenlafaxine, can optionally be employed in their free base form or as one of its salts and/or solvates thereof, where appropriate. Similarly, desvenlafaxine obtained according to Scheme 2 can be isolated in its free base form or as one of its salts or solvates thereof (e.g. desvenlafaxine succinate monohydrate).

Among other things, the invention includes:

Compound (III) obtained according to the process of the invention having a purity of at least approximately 98%, preferably of at least approximately 99%, more preferably of at least approximately 99.9%, as measured by HPLC.

Desvenlafaxine obtained according to the process of the invention having a purity of at least approximately 97%, preferably of at least approximately 99%, more preferably of at least approximately 99.8%, as measured by HPLC.

Desvenlafaxine succinate obtained according to the process of the invention having a purity of at least approximately 99%, preferably of at least approximately 99.9%, as measured by HPLC.

Desvenlafaxine succinate isolated from desvenlafaxine obtained according to the process of the invention having a particle size distribution wherein approximately 10% of the total volume (i.e., D₁₀ (v)) of particles having a diameter less than approximately 4 μM, approximately 50% of the total volume (i.e., D₅₀ (v)) of particles having a diameter less than approximately 41 μm, and approximately 90% of the total volume (i.e., D₉₀ (v)) of particles having a diameter less than approximately 155 μm.

Desvenlafaxine succinate isolated from desvenlafaxine obtained according to the process of the invention having a surface area of approximately 0.8457±0.0039 m²/g.

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

### SPECIFIC EXAMPLES

The following examples are for illustrative purposes only and are not intended, nor should they be interpreted to, limit the scope of the invention.

General Experimental Conditions:

i. HPLC Method

In the examples described below, the following analytical chromatographic HPLC method was used:

The chromatographic separation was carried out in a Kromasil 100 C8, 5 μm, 25 cm×4.6 mm. I.D. column at 40° C.

The mobile phase was prepared by mixing 2,000 g of 0.1 M (NH₄)H₂PO₄ buffer solution pH=6.0 (20:80) and 391.5 g of acetonitrile HPLC grade. The pH of the mixture should be adjusted, if necessary, to 6.4.

A (NH₄)H₂PO₄ buffer solution (0.1 M; pH=6.0) was prepared by dissolving 28.75 g of (NH₄)H₂PO₄ in 2,500 g of water and adjusting the pH=6.0 with H₃PO₄ or ammonium hydroxide.

The chromatograph was equipped with a 225 nm detector, and the flow rate was 1.2 mL per minute at 40° C. Test samples (20 μl) were prepared by dissolving the appropriate amount of sample to obtain 1 mg per mL concentration in the mobile phase.

ii. Particle Size Method

The particle size for desvenlafaxine succinate was measured using a Malvern Mastersizer S particle size analyzer with an MS1 Small Volume Sample Dispersion Unit stirred cell. A 300RF mm lens and a beam length of 2.4 mm were used. Samples for analysis were prepared by dispersing a weighed amount of desvenlafaxine succinate (approximately 0.1 g) in 20 mL of Lecithin Solution, previously prepared by dilution of 1.5 g of Soybean Lecithin to 200 mL of Isopar G. Mix gently until Lecithin dissolves (Lecithin solution). If it is necessary the solution can be sonicated. The suspension was delivered drop-wise to a background corrected measuring cell previously filled with dispersant (Isopar G) until the obscuration reached the desired level. Volume distributions were obtained for three times. Before and after the analysis, purge the sample and flush the MS1 unit with ethanol twice and with the dispersant at least three times. For characterization, the values of D₁₀, D₅₀ and D₉₀ (by volume) were specifically listed, each one being the mean of the six values available for each characterization parameter.

iii. Specific Surface Area Method

The BET (Brunauer, Emmett and Teller) specific surface area for desvenlafaxine succinate was measured using a Micromeritics ASAP2010 equipment. Samples for analysis were degassed at 30° C.-70° C. under vacuum for about two hours. The determination of the adsorption of N₂ at 77.3° K was measured for relative pressures in the range of 0.05-0.3 for a weighed amount of desvenlafaxine succinate (i.e., approximately 0.5 g).

iv. Residual Solvents (Gas Chromatography Method and Solutions)

Chromatographic separation was carried out in a VOCOL capillary column of 3 μm film thickness, 105 m×0.53 mm i.d. column. The chromatograph is equipped with a FID detector and a Head Space injection auxiliary device.

The oven temperature was programmed as follows: Initial 0-16 minutes 70° C., then the temperature is raised to 150° C. (ramp rate 25° C./minute) and is maintained at 150° C. for 3 minutes, then raised again to 240° C. with a ramp of 30° C. per minute. The injector and detector temperatures are then set at 220° C. and 250° C., respectively. Helium is used as carrier gas at a pressure of 20 psi with a flow of 10 mL/minute and a split ratio of 5:1 for Perkin Elmer 8700 and a split ratio of 3:1 for Agilent 6890N.

Samples were heated for 30 minutes at 85° C. in the head space device. After heating, the vials were pressurized with helium at 18 psi for 0.3 minutes. The sample loop was filled for 0.15 minutes (loop volume=1 mL) and then injected for 0.5 minutes.

Solutions:

Stock Solution of Acetone:

The stock solution of acetone was prepared so as to contain 1010 μg/mL of acetone in water by diluting a quantitatively known volume of acetone.

Standard Solution of Acetone:

The stock solution of acetone was diluted quantitatively with water to obtain solutions containing 0.15, 0.3, 0.5, 1, 2, 4, 10, 25, 51, 63, 101 and 126 μg/mL of acetone.

Test Solutions:

The test solutions were prepared by mixing approximately 100 mg of desvenlafaxine succinate in 5 mL of water.

Procedure:

The vials containing samples were sealed with suitable crimp caps and analyzed by head space using the above-described conditions.

### Example 1

### Preparation of 4-[2-amino-1-(1-hydroxycyclohexyl)ethyl] phenol Using a Selectride Solution

This example illustrates a process for converting Compound II into Compound III according to one aspect of the invention.

A 2 L flask at room temperature and under a nitrogen atmosphere was charged with 654 mL (124:33 g, 0.654 mol) of Selectride 1.0 M in THF solution. Then, a toluenic solution of 29.23 g (156.55 g, 0.117 mol) of 1-[2-amino-1-(4-methoxyphenyl)ethyl]cyclohexanol (i.e., Compound II) was added into the reaction mixture with stirring at room temperature. Next, the tetrahydrofuran was distilled under a nitrogen atmosphere between 79° C. to 110° C. The yellow suspension obtained was heated to reflux (approximately 115° C.) with continuous stirring and maintained at this temperature for 2 hours and 30 minutes.

Thereafter, the reactor contents were cooled to 10-15° C., and 300 mL of deionized water was added with continuous stirring. The temperature was then adjusted to 20° C., and the stirring was continued for 30 minutes. The resulting two phases were then acidified with hydrochloride acid (37%) to adjust the pH to about 1.0±0.2 (actual reading 0.99). The mixture was heated to reflux temperature and stirred at this temperature for 10 minutes. Then, the mixture was cooled to room temperature, and the phases were separated. The aqueous phase was washed twice with 81 mL (70.22 g) of toluene and then with 160 mL of heptane (109.44 g). The obtained aqueous phase was basified with sodium hydroxide with stirring to adjust the pH to between 10.3-10.5.

The resulting white suspension was stirred at room temperature for a minimum of 1 hour. Thereafter the suspension was filtered to give a white solid which was dried under vacuum at 60° C. to yield 24.73 g of 4-[2-amino-1-(1-hydroxycyclohexyl)ethyl]phenol (i.e., Compound III) (Yield: 89.63%; HPLC Purity: 98.181%, Assay: 92.31%).

### Example 2

### Preparation of 4-[2-amino-1-(1-hydroxycyclohexyl)ethyl] phenol Using Dodecanethiol

This example illustrates a process for converting Compound II into Compound III according to one aspect of the invention.

A 500 mL multi-necked flask, equipped with a mechanical stirrer and distillation equipment, was purged with nitrogen, and a nitrogen atmosphere was maintained. To the flask were added 1-[2-amino-1-(4-methoxyphenyl)ethyl]cyclohexanol (i.e., Compound II) (30 g; 0.120 mol) in toluene, 35.69 mL (0.144 mol) of dodecanethiol and PEG 400. Then, 33.50 mL (0.180 mol) of a methanol solution of sodium methanolate (30%) was added, and the reaction mixture was heated to 140° C. The methanol and the toluene were distilled off, and the suspension was stirred for 10 hours at 140-150° C. Thereafter, the temperature was lowered to about 45° C., and 70.8 mL of water was added.

Next, 103.3 mL of the 155 mL obtained suspension were charged into another reactor along with 28.32 mL of water and 56.64 mL (49.11 g) of toluene. The reaction mixture was then stirred for 15 minutes, and the resulting aqueous and organic phases were separated. The aqueous phase was next washed twice with 37.76 mL (32.74 g) of toluene. The pH of the aqueous phase was then adjusted to 2 with aqueous HCl. Thereafter, the acidified aqueous phase was heated to reflux for 10 minutes and was allowed to cool to room temperature. A yellow solution (141 mL) was obtained. Next, 32.25 mL of the solution was charged in another reactor, and the pH was adjusted to 6-7. Then, 3.5 mL (2.75 g) of 2-propanol was charged, and the yellow-orange solution was basified to pH 9.5 with sodium hydroxide 50%. The resulting suspension was stirred for approximately 1 hour at room temperature to produce a white suspension.

The white suspension was then filtered, and the solid was washed with 5 mL of water. The resulting wet solid was dried under vacuum at 60° C. until constant weight to yield 4 g (0.017 mol, 84.75%) of 4-[2-amino-1-(1-hydroxycyclohexyl)ethyl]phenol (i.e., Compound III) (HPLC Purity: 98.00%; Assay: 96.97%, Residue of Ignition: 0.31%).

### Example 3

### Preparation of Desvenlafaxine (o-desmethylvenlafaxine)

This example illustrates a process for converting Compound III into desvenlafaxine (i.e., Compound I) according to one aspect of the invention.

4-[2-amino-1-(1-hydroxycyclohexyl)ethyl]phenol (i.e., Compound III) (2.12 g; 0.009 mol) was suspended in 21.2 mL (16.79 g) of methanol under a nitrogen atmosphere. A white suspension was obtained. Then, 1.69 mL (0.022 mol) of formaldehyde (37%) was added, followed by 4.46 g (0.021 mol) of sodium triacetoxyborohydride added in portions. The resulting suspension was stirred for 4 hours and 40 minutes at room temperature. Then, 21.2 mL of water was added, and the stirring was continued at room temperature for 10 minutes. The methanol was removed by distillation, and the resulting solution was washed twice with 10 mL (8.67 g) of toluene. Thereafter, the pH was adjusted to 9.5 with sodium hydroxide 50%. The resulting suspension was cooled to 0-5° C. and was filtered. The wet solid was dried under vacuum at 60° C. until constant weight to yield 2.11 g (0.008 mol) of desvenlafaxine (HPLC Purity: 92.48%; Assay: 114.72%; Yield: 89.0%).

A 1.84 g portion of the desvenlafaxine obtained was then suspended in 9.2 mL of deionized water and 9.2 mL (7.28 g) of acetone. The suspension was stirred for 30 minutes at room temperature. Then, the suspension was heated to reflux and maintained at this temperature 5 minutes. The resulting suspension was cooled to 20-25° C. and was filtered. The wet solid was dried under vacuum at 60° C. until constant weight to yield 1.26 g (0.005 mol) of desvenlafaxine (HPLC Purity: 97.70%; Assay: 100.56%; Yield: 68.5%).

### Example 4

### Preparation of Desvenlafaxine (o-desmethylvenlafaxine)

This example illustrates a process for converting Compound III into desvenlafaxine (i.e., Compound I) according to one aspect of the invention.

Sodium triacetoxyborohydride (425 g; 0.020 mol) was suspended in 10 mL (8.72 g) of isopropyl acetate under a nitrogen atmosphere. A white suspension was obtained. Then, a solution of 1.5 mL (0.020 mol) of formaldehyde (37%) with 2.34 g (2 g dry) of wet 4-[2-amino-1- to (1-hydroxycyclohexyl)ethyl]phenol (i.e., Compound III) and 10 mL (8.72 g) of isopropyl acetate was added. The resulting suspension was stirred for 1 hour at room temperature. Then, 20 mL of water was added, and the stirring was continued at room temperature for 10 minutes. The resulting organic and aqueous phases were then separated, and the pH of the aqueous phase was adjusted to 9.5 with aqueous sodium hydroxide. Next, 2-propanol (10 mL; 7.85 g) was charged, and the resulting suspension was stirred at room temperature for two hours. The suspension was then filtered, and the wet solid was dried under vacuum at 40° C. until constant weight to yield 2.31 g (0.009 mol) of desvenlafaxine (HPLC Purity: 97.05%; Assay: 12237%).

A 1.06 g portion of the desvenlafaxine obtained above was then suspended in 6 mL of deionized water. Next, the suspension was heated to reflux and maintained at this temperature for one hour. The resulting suspension was cooled to 20-25° C. and was filtered. The wet solid was dried under vacuum at 40° C. until constant weight to yield 0.61 g (0.002 mol) of desvenlafaxine (HPLC Purity: 97.44%; Assay: 99.15%; Yield: 57.5%).

### Example 5

### Preparation of 4-[2-amino-1-(hydroxycyclohexyl)ethyl]phenol Using dodecanethiol

This example illustrates a process for converting Compound H into Compound III according to one aspect of the invention.

A suitable reactor and equipment was purged with nitrogen and a nitrogen atmosphere was maintained. Next, 1-[2-amino-1-(4-methoxyphenyl)ethyl]cyclohexanol hydrochloride (30 Kg; 105 moles) was suspended in 90 Kg of deionized water. The pH of resulting solution was then adjusted to 13 with sodium hydroxide (50%), and was stirred for 30 minutes. Then, 52 Kg (60 L) of toluene was charged to the reactor. The reaction mixture was then stirred for 30 minutes, and the resulting aqueous and organic phases were separated. The aqueous phase was then washed with 13 Kg (15 L) of toluene. Next, the combined organic extracts were treated with 25.5 Kg (30.2 L, 126 moles) of dodecanethiol and 59 Kg (52.4 L) of PEG 400. Then, 28.5 Kg (29.4 L, 158.3 moles) of a methanolic solution of sodium methanolate (30%) was added, and the reaction mixture was heated to 80° C. for 1 hour. The methanol and the toluene were then removed by distillation, and the suspension was stirred for 12 hours at 140±5° C. The temperature was then lowered to approximately 60° C., and 131 L of water and 57 Kg (65.7 L) of toluene were added. The reaction mixture was then stirred for 20 minutes at 45±5° C., and the resulting aqueous and organic phases were then separated. The aqueous phase was washed twice at 45±5° C. with 65.7 L (57 Kg) and 39.2 L (34 Kg) of toluene.

Next, n-butanol (30 Kg; 37 L) was charged over the aqueous phase and pH was adjusted to 9.5-10.0 with sodium hydroxide (50%) at room temperature. The resulting suspension was heated to reflux temperature and maintained at this temperature for 30 minutes. The resulting suspension was cooled to 20-25° C. and was filtered. 23.05 Kg of wet 4-[2-amino-1-(hydroxycyclohexyl)ethyl]phenol was obtained (Loss on Drying: 2.49%; Yield: 91%; HPLC Purity: 99.88%; Assay: 99.51%).

23.05 Kg of wet 4-[2-amino-1-(hydroxycyclohexyl)ethyl]phenol was suspended in 39 Kg (48.1 L) of n-butanol. The suspension was heated to reflux temperature and maintained at this temperature for 30 minutes. The resulting suspension was cooled to 20-25° C. and maintained at this temperature for one hour. Then the suspension was filtered and washed twice with 6 Kg of n-butanol and washed with 8 Kg of water. (Loss on Drying: 6.63%; Yield: 80.9%; HPLC Purity: 99.93%; Assay: 101.63%).

### Example 6

### Preparation of Desvenlafaxine (o-desmethylvenlafaxine)

This example illustrates a process for converting Compound III into desvenlafaxine (i.e., Compound I) according to one aspect of the invention.

21.4 Kg (91 moles) of wet 4-[2-amino-1-(hydroxycyclohexyl)ethyl]phenol was suspended in 87 Kg (99.8 L) of isopropyl acetate under a nitrogen atmosphere. The reaction mixture was cooled to 17-22° C., and 20.6 Kg (254 moles) of formaldehyde (37%) was added. The resulting solution was cooled at 0-5° C., and the aqueous phase was separated. Then, 3 Kg of anhydrous sodium sulfate was charged. In another reactor, 54 Kg (255 moles) of sodium triacetoxyborohydride was suspended in 87 Kg (99.8 L) of isopropyl acetate at 28±4° C. The anhydrous solution of isopropyl acetate was then added over the suspension of sodium triacetoxyborohydride and the temperature was maintained at 28±4° C. The reaction mixture was then stirred for 1 hour at 28 f 4° C.

Thereafter, 100 Kg of water was added in 45 minutes. The resulting organic and aqueous phases were then separated, and 39 Kg (49.7 L) of 2-propanol was charged into the aqueous phase. The aqueous reaction mixture was cooled at 20-25° C., and the pH was then adjusted to 9.5-10.0 with aqueous sodium hydroxide (50%). Then, the suspension was heated to reflux and maintained at this temperature for 30 minutes. The resulting suspension was cooled to 20-25° C., and the pH was checked again and corrected with hydrochloride acid (35%). The suspension was stirred at 20-25° C., and was filtered.

Next, 50.10 Kg of the wet desvenlafaxine obtained (Loss on Drying: 13.70%) was suspended in 168 Kg of deionized water. The suspension was heated to 90-95° C. and maintained at this temperature 30 minutes. The resulting suspension was cooled to and maintained at 20-25° C. for one hour and was filtered.

The wet desvenlafaxine obtained (38.2 Kg; loss on drying: 21.22%) was then suspended in 89 Kg (112.4 L) of methanol. The suspension was then heated to reflux temperature and maintained at this temperature 30 minutes. The resulting suspension was cooled to and maintained at 20-25° C. for one hour and was filtered (Loss on Drying: 0.77%; Yield: 93.20%; HPLC Purity: 99.72%; Assay: 100.65%).

A 21 Kg of wet desvenlafaxine obtained was suspended in 71 Kg (89.7 L) of methanol. The suspension was heated to reflux temperature and maintained at this temperature 30 minutes. The resulting suspension was cooled to 20-25° C., maintained one hour at this temperature and was filtered. The solid obtained was washed twice with 10 Kg water. (loss on drying: 19.93%; yield: 81.81%; HPLC Purity: 99.80%; assay: 100.16%).

### Example 7

### Preparation of Desvenlafaxine succinate monohydrate

This example illustrates a process for converting desvenlafaxine (i.e., Compound I) into desvenlafaxine succinate monohydrate according to one aspect of the invention.

Desvenlafaxine (21.30 Kg; 80.9 moles) was charged into a suitable reactor under nitrogen atmosphere with 9.1 Kg (77.06 moles) of succinic acid, 121 Kg (153 L) of acetone and 51 Kg of water. The suspension was heated to reflux temperature and maintained at this temperature 30 minutes. The resulting solution was cooled to 50-55° C. and was filtered.

Then, the solution was cooled to 30-35° C. and maintained for 3 hours at this temperature. Thereafter, the suspension was cooled to 20-25° C. and maintained at this temperature for 2 hours. Then, the suspension was filtered and washed twice with 6 Kg (7.6 L) of acetone. The wet solid (17.8 Kg) was dried under vacuum at 60±5° C. until constant weight to yield 17.80 Kg (44.56 moles) of desvenlafaxine succinate, which was subsequently milled and sieved (HPLC Purity: 99.9%; assay: 100.2%; yield: 68.8%).

The desvenlafaxine succinate obtained by the processes of the invention typically has the following particle size distribution: D₁₀ (v): 3.0 to 4.0 μm; D₅₀ (v): 35.0 to 41.0 μm; D₉₀ (v): 140.0 to 155.0 μm; and typically has the following surface area: 0.8457±0.0039 m²/g.

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

What is claimed is:

1. A process for preparing desvenlafaxine free base and salts or solvates thereof comprising N,N-dimethylating 4-[2-amino-1-(hydroxycyclohexyl)ethyl]phenol (Compound III) to obtain desvenlafaxine free base.

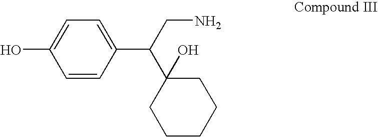

2. The process of claim 1, further comprising converting said desvenlafaxine free base into salts or solvates thereof.

3. The process of claim 2, wherein said reductive methylation comprises treating Compound III with formaldehyde and sodium triacetoxyborohydride in at least one organic solvent.

4. The process of claim 3, wherein said at least one organic solvent is at least one of a lower alcohol, an ester solvent and combinations thereof.

5. The process of claim 3, wherein said at least one organic solvent is at least one of methanol, isopropyl acetate and combinations thereof.

6. The process of claim 3, wherein said at least one organic solvent is isopropyl acetate.

7. The process of claim 3, further comprising at least one of (i) adding water to the reaction mixture, (ii) extracting the reaction mixture to form at least one organic phase and at least one aqueous phase, (iii) basifying the aqueous phase to an appropriate pH to obtain desvenlafaxine free base, (iv) isolating desvenlafaxine free base and (v) converting desvenlafaxine free base into a salt.

8. The process of claim 7, wherein said step of basifying the aqueous phase to an appropriate pH to obtain desvenlafaxine free base comprises adjusting the pH to between approximately 9.5 to approximately 10.0 with at least one base.

9. The process of claim 8, wherein said at least one base is at least one inorganic base.

10. The process of claim 8, wherein said at least one base is at least one hydroxide base.

11. The process of claim 8, wherein said at least one base is sodium hydroxide.

12. The process of claim 1, wherein said desvenlafaxine free base and salts or solvates thereof has a purity of at least approximately 97% as measured by HPLC.

13. The process of claim 12, wherein said desvenlafaxine free base and salts or solvates thereof has a purity of at least approximately 99% as measured by HPLC.

14. The process of claim 13, wherein said desvenlafaxine free base and salts or solvates thereof has a purity of at least approximately 99.8% as measured by HPLC.

15. The process of claim 1, wherein said desvenlafaxine free base and salts or solvates thereof is desvenlafaxine succinate salt.

16. The process of claim 15, wherein said desvenlafaxine succinate salt is desvenlafaxine succinate monohydrate.

17. The process of claim 15, wherein said process further comprises
i. treating said desvenlafaxine free base with succinic acid in acetone and water;
ii. heating the mixture of step i. to reflux;
iii. isolating desvenlafaxine succinate salt; and
iv. drying said desvenlafaxine succinate salt.

18. The process of claim 15, wherein said desvenlafaxine succinate salt has a purity of at least approximately 99%, as measured by HPLC.

19. The process of claim 18, wherein said desvenlafaxine succinate salt has a purity of at least approximately 99.9%, as measured by HPLC.

20. The process of claim 17, further comprising milling and sieving said desvenlafaxine succinate salt.

21. The process of claim 20, wherein said desvenlafaxine succinate salt has a particle size distribution D₁₀ (v) of approximately 4 μm.

22. The process of claim 20, wherein said desvenlafaxine succinate salt has a particle size distribution D₅₀ (v) of approximately 41 μm.

23. The process of claim 20, wherein said desvenlafaxine succinate salt has a particle size distribution D₉₀ (v) of approximately 155 μm.

24. The process of claim 20, wherein said desvenlafaxine succinate salt has a surface area of approximately 0.8457±0.0039 m²/g.

25. A process for preparing 4-[2-amino-1-(hydroxycyclohexyl)ethyl]phenol (Compound III) 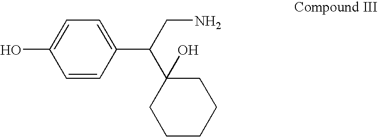
comprising O-demethylating 1-[2-amino-1-(4-methoxyphenyl)ethyl]cyclohexanol (Compound II)

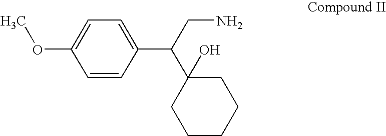

26. The process of claim 25, wherein said O-demethylating comprises treating Compound II with dodecanethiol.

27. The process of claim 26, wherein said O-demethylating comprises reacting a mixture of Compound II, dodecanethiol, and at least one organic solvent in the presence of sodium methanolate.

28. The process of claim 27, wherein said at least one organic solvent is at least one ether solvent.

29. The process of claim 27, wherein said at least one organic solvent is at least one polyether solvent.

30. The process of claim 27, wherein said at least one organic solvent is polyethylene glycol.

31. The process of claim 27, further comprising heating said mixture for approximately 12 hours at approximately 140° C.

32. The process of claim 27, further comprising at least one of:
i. adding water to said mixture;
ii. extracting said mixture to obtain at least one organic phase and at least one aqueous phase;
ii. basifying said at least one aqueous phase to a pH appropriate to obtain Compound
iii. isolating Compound III;
iv. purifying Compound III; and
v. drying Compound III.

33. The process of claim 32, wherein said basifying comprises adjusting the pH to between approximately 9.5 to approximately 10.0 with at least one base.

34. The process of claim 33, wherein said at least one base is at least one inorganic base.

35. The process of claim 34, wherein said at least one inorganic base is at least one hydroxide base.

36. The process of claim 35, wherein said at least one inorganic base is sodium hydroxide.

37. The process of claim 25, wherein said O-demethylating comprises treating Compound II with at least one L-selectride.

38. The process of claim 37, wherein said process comprises reacting a mixture of Compound II with at least one L-selectride in at least one organic solvent.

39. The process of claim 38, wherein said at least one organic solvent is at least one non-polar solvent.

40. The process of claim 38, wherein said at least one organic solvent is a hydrocarbon solvent.

41. The process of claim 38, wherein said at least one organic solvent is toluene.

42. The process of claim 38, further comprising heating said mixture for approximately 2 hours at approximately 115° C.

43. The process of claim 38, further comprising at least one of:
i. adding water to said reaction mixture;
ii. extracting said mixture to obtain at least one organic phase and at least one aqueous phase;
iii. basifying said aqueous phase to a pH appropriate to obtain Compound III;
iv. isolating Compound III;
v. purifying Compound III; and
vi. drying Compound III.

44. The process of claim 43, wherein said basifying comprises adjusting the pH to between approximately 9.5 to approximately 10.0 with at least one base.

45. The process of claim 44, wherein said at least one base is at least one inorganic base.

46. The process of claim 45, wherein said at least one inorganic base is at least one hydroxide base.

47. The process of claim 45, wherein said at least one inorganic base is sodium hydroxide.

48. The process of claim 25, wherein said Compound III has a purity of at least approximately 98% as measured by HPLC.

49. The process of claim 48, wherein said Compound III has a purity of at least approximately 99% as measured by HPLC.

50. The process of claim 49, wherein said Compound III has a purity of at least approximately 99.9% as measured by HPLC.

51-54. (canceled)
